(19) United States
(12) Patent Application Publication
(10) Pub. No.: US 2020/0289304 A1
(43) Pub. Date: Sep. 17, 2020

(54) WRIST SUPPORT AND METHODS OF USE

(71) Applicant: V-Align, LLC

(72) Inventors: Sundaram Ravikumar, Briarcliff Manor, NY (US); Harry Allan Alward, Shelton, CT (US)

(73) Assignee: V-Align, LLC, Dobbs Ferry, NY (US)

(21) Appl. No.: 16/084,359

(22) PCT Filed: Jan. 19, 2017
(86) PCT No.: PCT/US2017/014194

§ 371 Date: Sep. 12, 2018

(51) Int. Cl. A61F 5/37; A61G 13/12; A61M 5/52

(52) U.S. Cl. CPC A61F 5/3761; A61M 5/52; A61G 13/1235; A61G 13/124

(57) ABSTRACT

A wrist support apparatus capable of positioning and restraining a patients wrist including an elongated base member and an adjustable means to adjust the angle of the patient's exposed wrist for insertion of a medial line such as a radial arterial line or intravenous line. The wrist support apparatus includes a first fastening means to restrain the patient's fingers, a second fastening means to restrain the patient's forearm and a third fastening means to restrain the patient's thumb. A fourth fastening means may be used to secure the wrist support apparatus to an object such as a table. A method of use includes positioning the patient's wrist at an angle during the insertion of the medical line and may include an adjustable means for such positioning.

### RELATED APPLICATIONS

This application claims priority from provisional application Ser. No. 62/105,164 filed Jan. 19, 2016 and provisional application Ser. No. 62/422269 filed Nov. 15, 2016.

### FIELD OF THE INVENTION

This invention relates generally to a wrist support apparatus and method of use, more specifically for methods of maintaining a patients wrist in an extended position during radial artery procedures such as cannulation of a catheter and/or arterial blood sampling.

### BACKGROUND OF THE INVENTION

Arterial access and insertion of a cannula is a common procedure in various critical care settings and is known as arterial cannulation. Arterial line placement may be made in a number of arteries such as the radial, ulnar, brachial, axillary, posterior tibial, femoral, and dorsalis pedis arteries.

One procedure utilizing arterial cannulation is intra-arterial blood pressure measurement which is considered more accurate than measurement of blood pressure via a conventional pressure cuff, on the surface of the upper arm of a patient. The blood pressure of critically ill patients is typically monitored in this fashion. Intra-arterial blood pressure management permits the rapid recognition of any small change in the patient's blood pressure. Such constant monitoring of blood pressure is needed for critically ill patients as well as those patients who are receiving vasoactive drugs.

Arterial cannulation is also employed for repeated arterial, blood gas samples to be drawn without further injury to the patient following initial insertion of the arterial cannula. In such procedure a port may be inserted through the cannula and left in the patient's skin for a period of time so as to provide access to the target artery. Another utilization of a radial artery cannulation is during C.O.P.D. therapy, which therapy may be performed in the patients home outside of a medical surrounding.

Further, arterial cannulation may be used during the medical procedure of a cardiac catheterization. In cardiac catheterization a catheter is threaded through an artery or vein up into the heart for various procedures such as angioplasty or placing a stent inside a vessel. This procedure may be performed using the artery access point of the leg or arm.

One common access point for arterial cannulation is the femoral artery in a patient's groin area of the leg. Accessing the femoral artery has many problems such as bleeding at the incision access point and nerve damage. Further, following the procedure the patient must maintain stillness in a prone position for at least 5 hours to ensure the access point does not bleed.

A second common access point is the radial artery which is located in the patient's forearm and accessible at the wrist. The radial artery is easier to access due to its proximity to the surface of the arm. It is also smaller than the femoral artery and thus it is easier to apply direct pressure to the access point after conclusion of the procedure in order to stop bleeding upon removal of the catheter and other equipment. Further, radial artery access typically does not cause as much pain and discomfort for patients compared to access via the femoral artery. Further, based on the smaller artery and location on the wrist the patient need not stay in bed lying prone for 5 hours after the conclusion of the cardiac catheterization procedure, resulting in a shorter port-operative period.

Repeated incisions or punctures to the wrist area and possible damage to the radial artery must be avoided so as to reduce pain to the patient as well as complications, infection and side effects. Further, it is important in any intra-arterial therapy that the patient's wrist be properly oriented in an angled position to easily receive and start the arterial or intravenous line.

There are several known techniques for accessing the radial artery for arterial cannulation, insertion of a line, or arterial catheter placement. During such procedures it is very important to maintain the correct extension and flexion of the wrist so as to access the radial artery. While the radial artery is closer to the surface of the skin than the femoral artery, it is still not as close to the skin as for example a vein. Thus there exists a need to better extend the wrist of a patient so as to access the radial artery. By elevating and extending the wrist of a patient the radial artery is more easily accessible for incision or puncture and it is also better stabilized in the tissue of the patient's wrist and forearm. Therefore, the doctor or other medical professional has easier access to the radial artery resulting in a quicker and more accurate arterial cannulation, which may reduce or eliminate multiple attempts and punctures, as well as complications. The same procedure and needs are known and recognized, in the intravenous process for an IV line insertion,

The skin overlying the radial artery is very sensitive and the artery has a Is tendency to spasm as cannulation is made. Therefore, the wrist area needs to be restrained at the proper angle and flexion position to reduce the chance of improper insertion of a cannula or the catheter due to the patient's voluntary or involuntary movements.

The cannula, including a catheter and needle combination, should be inserted into the patient's radial artery at the wrist at a 25°-50° angle to the skin in the long plane of the artery. This insertion should be performed slowly and deliberately to maintain the proper angle of insertion. Typically, the natural reflexes in the wrist area of the patient and resulting spasms in the radial artery may compromise the cannulation so such reflexes and spasm must be constrained to the extent possible during the procedure.

Notably, maintaining the position and extension of the wrist and avoiding catheter movement continue after initial insertion of the cannula and catheter throughout the entire procedure such as an angioplasty. Wrist movements, even small movement, may contribute to decannulation of the intra-arterial line whether the procedure is an angioplasty or continuous blood pressure monitoring. Decannulation of the intra-arterial line may cause the blood to flow out of the radial artery at systolic pressure, which in a worst case scenario if left undetected and untreated may cause a person to bleed to death. In any case the application of immediate pressure to the site of the intra-arterial line and decannulation must be made to reduce or eliminate the occurrence of a large hematoma that would directly occlude the affected artery. To avoid this, the arterial line flange or adaptor is typically taped to the skin to prevent the line from becoming dislodged. However, the need for additional securement, and stability of the wrist position is necessary to prevent movement in order to avoid tissue erosion of the artery.

Traditional methods of extending the wrist and stabilizing the wrist and arm during radial artery cannulation and the following procedures or period of time has been made via tape, rolled up towels and possibly an arm board. None of these apparatus, devices or method work over time. optimally or consistently. Thus a need exists for a wrist support for use during radial arterial cannulation and during any related is medical procedure employing a radial arterial line.

In the past, numerous materials have been used to extend, elevate and stabilize the wrist of a patient during arterial cannulation including gauze, tape, or towels. These materials were used to adjust, extend and maintain the patient's wrist to the proper extended position, Conventionally gauze or towels or other padding materials have been rolled up into a cylinder, then placed and possibly taped onto a straight board or even the operating table itself to obtain a suitable angle for the wrist. The arm itself or a portion of the upturned palm or fingers may be taped to the arm board or operating table to partially stabilize the arm in the upturned position with the wrist extended in the upturned position. Once the cannula has been inserted into the radial artery it is then taped to the skin surface of the patient during the following medical procedure such as monitoring the blood pressure or a cardiac catheterization. Such taping of the arterial line does not provide adequate support, restraint or stabilization of the arterial line.

In certain instances, the rolled up padding materials under the extended wrist may be removed once the arterial line has been inserted. Removal of the wrist support may increase the risk of decannulation and other complications related to the inserted radial artery line.

Further, the prior techniques are tedious and time consuming thus taking up the time of valuable hospital personnel who would otherwise be free to perform other duties. As stated above, the materials used to position the wrist are not stable and the methods used do not ensure an accurate angle of the extended wrist, or maintenance of the angle and extension following the cannulation and insertion of the line.

Additionally, such conventional prior art apparatus, materials and methods do not properly position the patient's wrist to receive the redial arterial line and can be uncomfortable for the patient. For instance, the adhesive tape may cause skin reactions, rashes, pain and has a tendency to abrade the hair on the patient's arm.

Further, the combination of the rolled up towel or gauze and tape does not provide support, restraint or stabilization of the extended wrist at a set angle resulting in movement of the patient's wrist and/or arm. The patient's fingers, hand and wrist may have reflexed movement and the rolled up materials under the wrist do not maintain or stabilize the positioning of the extended wrist. During such movement there is also a risk of decannulation of the arterial line. After decannulation the patient must endure a repeated cannulation along with other complications such as a hematoma, blood loss, pain, infection and extended post-operative time and impairments.

To avoid complications from commonly used adhesive tape on the patient's skin surface some prior art arm boards and other wrist supports have included a strap as a fastener. Such arm boards or other wrist supports known to those of skill in the art are not optimum in that they do not obtain and maintain a proper angle for radial artery cannulation and for all medical procedures following such cannula insertion. Notably, the thumb is not hyperextended on its own so as to provide a better angle of extension of the entire wrist. While not being bound by theory, it is contemplated that by hyperextending the thumb separately from the remaining four fingers and upturned palm the radial artery is better positioned and exposed at an angle for insertion of the cannula and better maintained in a secure manner throughout the medical procedure. Thus a need exists for a wrist support which includes hyperextension of the thumb separate from the fingers and palm of the hand during radial artery carmulation and any subsequent medical procedure.

Known arm boards and other arterial wrist supports are comprised of wood or metal and may be uncomfortable to the patient when in use. Thus a need exists for a portion of the support apparatus to be comfortable to the patient while still maintaining the stability of the extended wrist, hand and thumb. Further, a need exists for a wrist support apparatus where the support may have a varied angle or support level such that the wrist support is capable of adjustment such as inflation and deflation to vary the angle of wrist extension. A further need exists for a wrist support apparatus to position and restrain the wrist of a patient for use in insertion of an intravenous line.

A further need exists for a wrist support apparatus which is stable on various surfaces such as an operating table, hospital bed, kitchen table and other surfaces. For instance, where a patient is undergoing C.O.P.D. therapy in a home a medical professional may perform radial artery cannulation on various surfaces of a table, bed chair or other furniture.

Another need exists for a wrist support apparatus which secures the inserted radial artery line to the patient during a medical procedure.

A further need exists for a wrist support apparatus which is adjustable and the angle of support can vary based on the level of the wrist support apparatus.

A further need exists for a wrist support apparatus which is inflatable and the angle of support can vary based on the inflation level of the wrist support apparatus.

A continuing need exists for an economic, convenient and effective wrist support apparatus for supporting a patient's wrist and forearm for such therapy.

These and other needs are met by the present invention including a wrist support apparatus and method of use. Other advantages of the present, invention will become apparent from the following description and appended claims.

### SUMMARY OF THE INVENTION

This invention provides an apparatus for supporting a patient's wrist, more specifically when the patient is undergoing a radial arterial cannulation or intravenous line insertion. The invention further provides an apparatus for securing an arterial line during a medical procedure or therapy. The invention further provides for a method of use including arterial cannulation. The invention includes an adjustable wrist support.

### BRIEF DESCRIPTION OF THE DRAWINGS

FIG. 1 is a perspective view of one embodiment of a wrist support of the present invention including an inflatable means.

FIG. 2 is another embodiment of the present invention of FIG. 1 including a further fastening means to secure the inventive inflatable wrist support to a board or table or other location.

FIG. 3 is another embodiment of the present invention of FIG. 1 including a manual pump as part of the inflatable means.

FIG. 4 is a bottom view of the embodiment of a wrist support according to FIG. 1 including a non-skid member.

FIG. 5 is is a perspective view of one embodiment of an inflatable wrist mean with the wrist support in use with an arm of a patient and showing a radial arterial line in use with the present invention.

FIG. 6 is a perspective view of another embodiment of a wrist support of the present invention.

FIG. 7 is a top view of the embodiment of a wrist support of the present invention as shown in FIG. 6.

FIG. 8 is side view (from proximal end) of the embodiment of a wrist support of the present invention as shown in FIG. 6 including a foam portion.

FIG. 9 is a bottom view of the embodiment of a wrist support of the present invention as shown in FIG. 6 including a non-skid member.

FIG. 10 is a perspective side view of an embodiment of a wrist support of the present invention not including any fastening means.

FIG. 11 is a bottom view of the base member of the embodiment of a wrist support of the present invention as shown in FIG. 6 without any fastening means attached.

FIG. 12 is an exploded bottom perspective of a fastening means attached to a tab on the bottom of the base member of one embodiment of a wrist support of the present invention.

FIG. 13 is a perspective view of the embodiment of a wrist support of the present invention as shown in FIG. 6 in use on an arm of a patient and showing a radial arterial line in use with the present invention.

FIG. 14 is a perspective view of a third embodiment of a wrist'support of the present invention including a third fastening means.

FIG. 15 is a top view of the embodiment of a wrist support of the present invention as shown in FIG. 14.

FIG. 16 is a perspective view of the embodiment of a wrist support of the present invention as shown in FIG. 6 in use on an arm of a patient and showing an intravenous line in use with the present invention.

### DETAILED DESCRIPTION OF THE INVENTION

The invention is a wrist support apparatus including an elongated base member with a proximal end and a distal end and a having a multitude of apertures for insertion of fastening means. The elongated base member is attached to an upper member which may be adjustable which may be adjustable in one embodiment and may also be inflatable in an embodiment and may also be non-adjustable in a sloped shape. The upper member may be an inflatable means which may be a bladder, with the inflatable means having a proximal end and a distal end coinciding to the proximal end and the distal end of the elongated base member. The proximal end of the inflatable means supports the forearm immediately above the wrist when in use. The distal end of the inflatable means supports the palm of the hand when in use. The inflatable means supports the wrist of a patient in an extended position when the hand is faced upward. The sloped member in another embodiment may have a proximal end and a distal end coinciding to the proximal end and the distal end of the elongated base member with the proximal end of the sloped member is sloped upward from the beginning of the proximal end toward the distal end and supports the forearm immediately above the wrist. The distal end of the sloped member is also sloped upward from the distal end toward the proximal end at a slope angle higher than that of the slope of the proximal end toward the distal end. The middle portion of the sloped member forms an angle similar to an inverted “\/” based on the meeting of the ramped is slopes from the proximal end and the distal end of the sloped member. The middle portion of the sloped member supports the wrist of a patient in an extended position when the hand is faced upward. The distal portion of the sloped member supports the palm and fingers of the patient when in use. The distal end of the elongated base member includes at least one aperture for attachment to a fastening means so as to secure the thumb of the patient when in use. The distal end of the elongated base member may further include another aperture for attachment to a fastening means so as to secure the extended fingers of the patient when in use. The proximal end of the elongated member may include a fastening means within the at last two apertures to secure and stabilize the forearm of the patient.

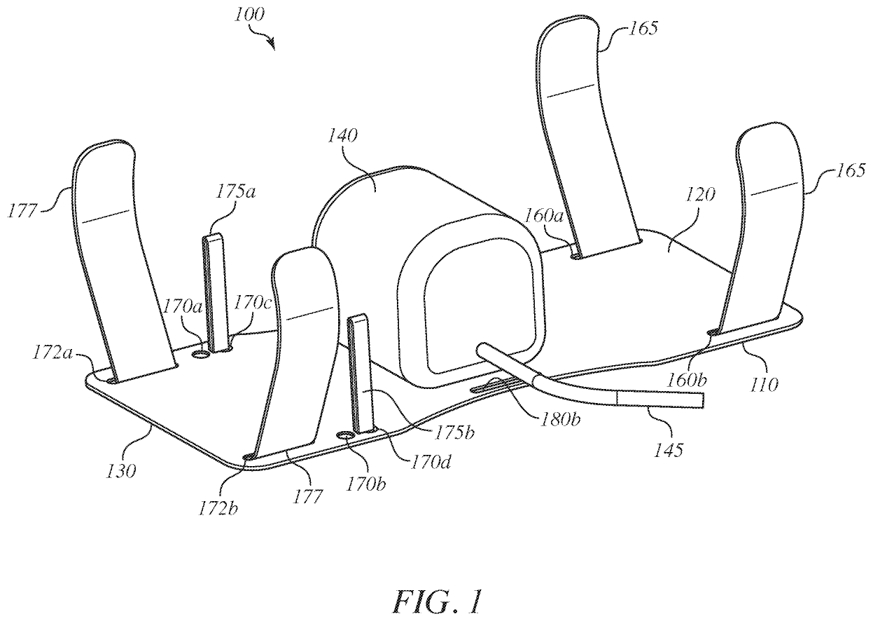

FIG. 1 shows one embodiment of the inventive wrist support 100 which in this embodiment includes an adjustable and inflatable upper member such as an air bladder. Turning to FIG. 1 the inventive inflatable wrist support includes an elongated base member 110 having a proximal end 120 and a distal end 130. The proximal end includes at least two apertures 160a, 160b for insertion of a fastening means 165 to secure the forearm of a patient when the inventive inflatable wrist support is in use. The distal end 130 includes at least, one aperture 170a, 170b for attachment to a fastening means 175a, 175b to secure the thumb of a patient when the inventive inflatable wrist support is in use, The distal end 130 may also include a second set of apertures 170c, 170d in order to secure or tie the fastening means 175a, 175b. The distal end 130 further includes at least one aperture 172a, 172b for attachment to a fastening means 177 to secure the fingers of a patient when the inventive inflatable wrist support is in use. The elongated base member 110 may be comprised of any rigid material sufficient to support the arm and hand placed upon it. Suitable materials include without limitation polymers, plastics, foam, wood, metals and the like. The elongated base member 110 is preferably comprised of a polymer or plastic material for economic reasons and to make the inventive inflatable wrist support 100 easy to use by a medical professional when in operation. For commercialization and mass production, the inventive inflatable wrist support 100 may formed, of a suitable disposable material. In other embodiments the inventive inflatable wrist support 100 may be comprised a suitable rigid material capable of being sterilized and reusable, or capable of autoclaving, and in this embodiment the fastening means 165, 175, 177 would be disposable and replaced after the sterilization of the previously used elongated base member 110 and inflatable means 140. In another embodiment the elongated base member 110 may be comprised of a semi-rigid material.

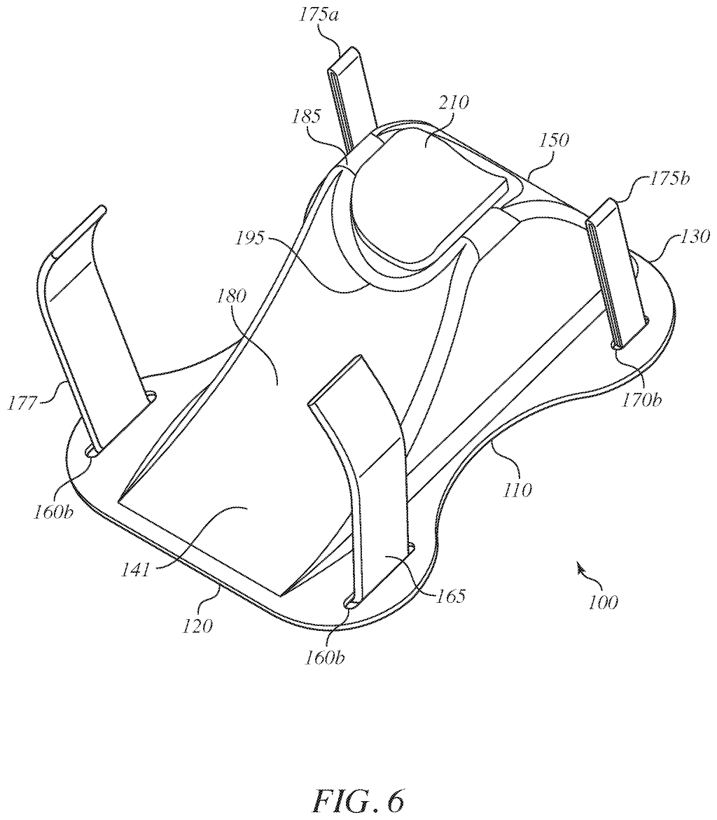

The elongated base member 110 may be a shape suitable to be stable when placed on surface such as a table or medical surface. In one embodiment as shown in FIG. 1 the shape of the elongated base member 110 is a rectangle with curved portions near the center on the long side of the rectangle. In another embodiment as shown in FIG. 6 the shape of the elongated base member 110 is a square or rectangle with curved portions near the center on the long side of the elongated base member 110. Other shapes may be employed such as oval, sphere, nephroid and any other geometric shape or combination thereof. In the embodiment shown in FIG. 1 the inflatable means 140 is located equidistant form the edge of the proximal end 120 of the elongated base member 110 and the distal end 130 of the elongated base member 110 but in other embodiments the length from the center of the elongated base member 110 to the edge of the proximal end 120 of the elongated base member 110 may be a different length than to the edge of distal end 130 of the elongated base member 110 in that the elongated base member 110 may not be symmetrical and even on each end from the center point. In other embodiments the ends (120, 130) of the elongated base member 110 may be different shapes, by way of example only, the proximal end 120 of the elongated base member 110 may be tapered inward or shaped like a rectangle while the distal end 130 of the elongated base member 110 may be shaped like a square or semi-circle. Again combinations of shapes may be used on each end (120, 130) as well as for the center potion of the elongated base member 110.

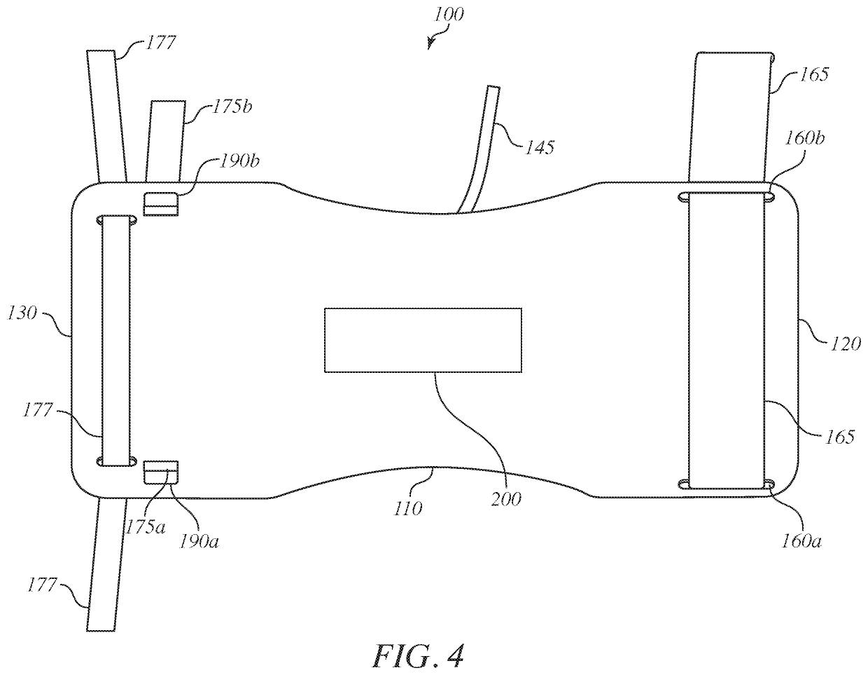

The bottom of the elongated base member 110 may optionally include, as seen in FIG. 4, an attached non-skid member 200 which acts to stabilize the inventive inflatable wrist support 100 when placed on a metal surface, wood surface, plastic surface and other known surfaces. The non-skid member 200 may be attached to the bottom of the elongated base member 110 by an adhesive or other known fastening means.

In this embodiment of the present invention, the elongated base member 110 also includes at least two apertures 160a, 160b at the proximal end 120. A fastening means 165 is inserted within each of the apertures 160a, 160b and is looped under the bottom of the inventive inflatable wrist support 100 as shown in FIG. 1. Each end of the second fastening means 165 is located above the top of the elongated base member and in, use fastens together to secure the forearm of the patient when in use. The fastening means 165 should provide a secure fit and restraint of the patient's forearm. One example of the fastening means 165 may be a strap with Velcro fasteners on each end when joined together to secure the forearm of the patient. The fastening means 165 may be other known straps, an elastic band, a belt, or the like. The fastening means 165 may be a combination of a flexible material with buckles, snaps Velcro of any commonly known fastener or locking means. Those skilled in the art may employ other known fastening means to secure and stabilize the forearm of the patient when the inventive inflatable wrist support 100 is in use for radial arterial cannulation or intravenous insertion. Stabilization is important to control the artery and wrist during the cannulation as well stabilizing the inserted radial arterial line during any subsequent medical procedure.

The elongated base member 110 also includes at least two apertures 172a, 172b at the distal end 130. A fastening means 177 is inserted within each of the apertures 172a, 172b and is looped under the bottom of the inventive inflatable wrist support 100 as shown in FIG. 1. Each end of the second fastening means 177 is located above the top of the elongated base member and in use fastens together to secure the extended fingers of the patient when in use. The fastening means 177 should provide a secure fit and restraint of the patient's fingers when in use. One example of the fastening means 177 may be a strap with Velcro fasteners on each end when joined together to secure the fingers of the patient. The fastening means 177 may be other known straps, an elastic band, a belt, or the like. The fastening means 177 may be a combination of a flexible material with buckles, snaps Velcro of any commonly known fastener or locking means. Those skilled in the art may employ other known is fastening means to secure and stabilize the fingers of the patient when the inventive inflatable wrist support 100 is in use for radial arterial cannulation. Stabilization is important to control the artery and wrist during the cannulation as well as stabilizing the inserted radial arterial line during any subsequent medical procedure. In other embodiments the fastening means to stabilize the fingers could be a glove attached to the distal end 130 of the elongated base member 110 such that the patient inserts fingers within the glove and the fingers are thus stabilized. Any know stabilization or fastening means to stabile or restraint the fingers may be employed in the inventive wrist support 100.

A further fastening means 175a, 175b may be used to secure the thumb of the patient when in use. The fastening means 175a, 175b may be an elastic material such as rubber constraints such as rubber bands, which are hooked individually onto each of the tabs 190a, 190b (shown in FIG. 4). In another embodiment of the present invention only one aperture 170a is used with one tab 190a and one fastening means 175a or in the alternative aperture 170b is used with one tab 190b and one fastening means 175b the choice depending on the right or left hand of the patient such that the one fastening means (either 175a or 175b) secures and hyperextends the thumb of the patient. Such choice will depend on whether the radial arterial cannulation is being performed on the right wrist or left wrist. It is preferred that the elongated base member 110 include two apertures 170a, 170b so that the inventive inflatable wrist support 100 is universal and can be used with both the right hand wrist and left hand wrist. The distal end 130 may also include a second set of apertures 170c, 170d in order to secure, loop or tie the fastening means 175a, 175b. In one embodiment, the fastening means 175a is a rubber band and is inserted into aperture 170a from the top and looped under the bottom of the elongated base member 110 and up through aperture 170c and then tied in a knot by looping one end of the fastening means 175a through the other open end of the fastening means 175a and pulled tight. The order can be reversed with the fastening means 175a being inserted into aperture 170a or 170c from the bottom of the elongated base member 110. The same method or other known methods may be employed with apertures 170b, 170d and fastening means 175b. Other known fastening means 175a, 175b include straps, loops and the like. Those skilled in the art may employ other known fastening means to secure and hyperextend the thumb of the patient when the inventive inflatable wrist support 100 is in use for radial arterial cannulation with the palm facing upward.

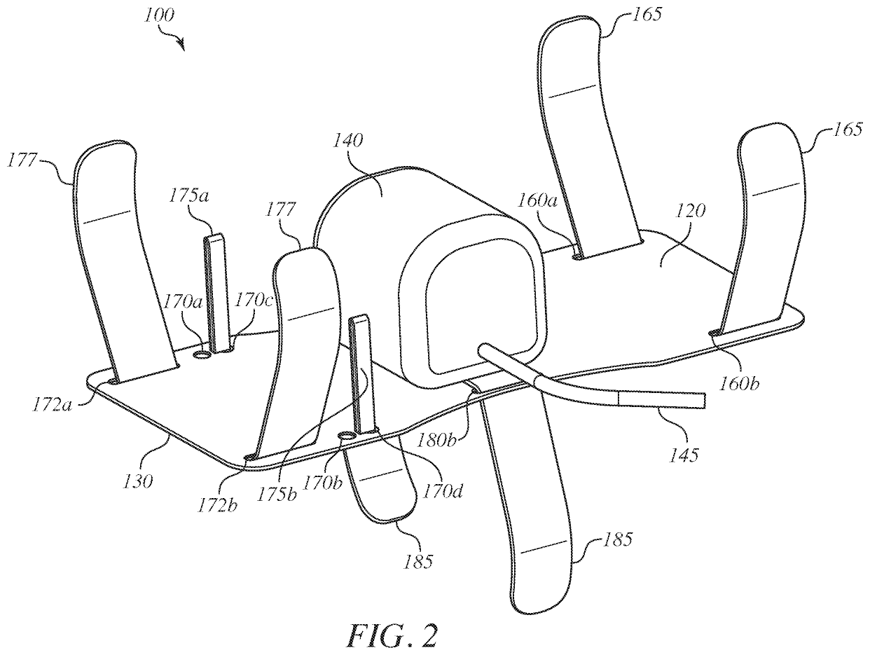

The elongated base member 110 is attached to an inflatable means 140. The inflatable means 140 may be slightly smaller in length and width than the elongated base member 110 for stability of the inventive inflatable wrist support 100 as well as providing for the apertures (160a, 160b, 170a, 170b) as seen in FIGS. 1 and 2. The inflatable means 140 supports the wrist when in use. The inflatable means 140 may be any known component capable of inflation. The inflatable means 140 may be adjustable based on the size of the wrist to be supported or the user's required angle to conduct the radial arterial cannulation. The inflatable means 140 may be inflated to a level and after the initial radial arterial cannulation reduced while a drug is being administered or for instance during C.O.P.D. therapy. The inflatable means 140 may be inflated, deflated, inflated and so on many times throughout a patient's session as each inflation or deflation may adjust the degree of angle of the wrist.

The inflatable means 140 may be a semi-rigid or a flexible material capable of supporting a patient's wrist when in use. The inflatable means 140 may be a bladder such as an air bladder. The air bladder may be realized by two plastic films that are joined together preferably by heat or RF welding to form an air chamber therebetween. The inflatable means 140 when an air bladder can be manually inflated by a pump (e.g., a standard syringe barrel) that is interfaced thereto via a valve assembly. The valve assembly may be a luer having an internal valve which allows for inflation and deflation. Alternatively, both separate inflation and deflation valves may be provided. In yet another alternative, the inflation/deflation valve(s) may be omitted and the inflatable means 140 when an air bladder is pre-filled with air at the desired pressure during manufacture.

The inflatable means 140 when an air bladder may be comprised of any material compatible to the skin of the user such as a polymer, film, laminated polymer, nonwoven or other mater or combinations thereof. The material of the inflatable means 140 when an air bladder must be flexible so as to move between an inactivated to activated state and return, as well as support the wrist being elevated such that it is partial or semi-rigid. One possible material is a polyurethane film or polymer. The inflatable means 140 when an air bladder is connected to the elongated base 110 by a connecting means (not shown). The connecting means may be any permanent fastening means such as glue, weld, or RF bonding or may be a removable fastening means such as a polymer or plastic layer connected to Velcro.

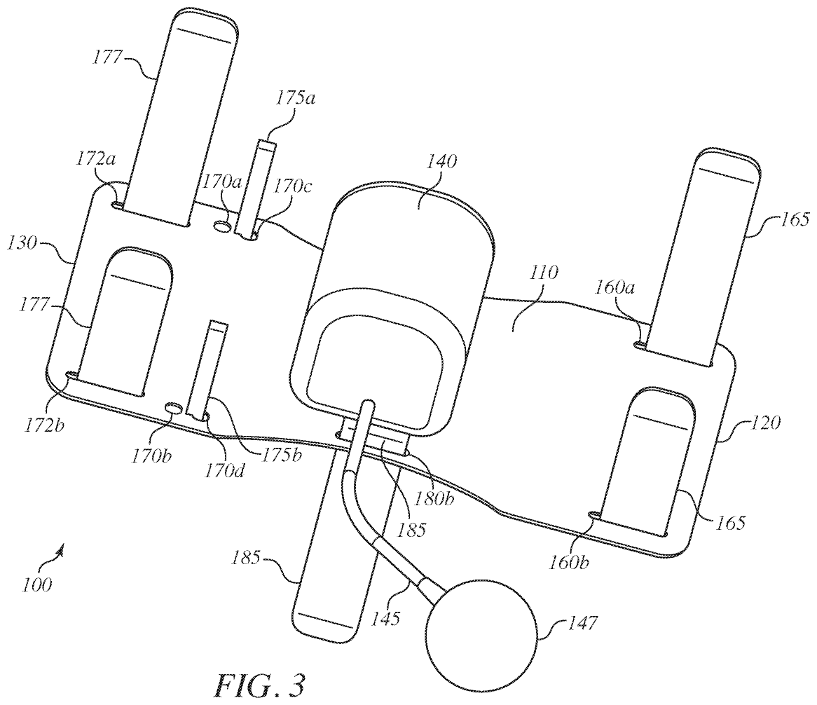

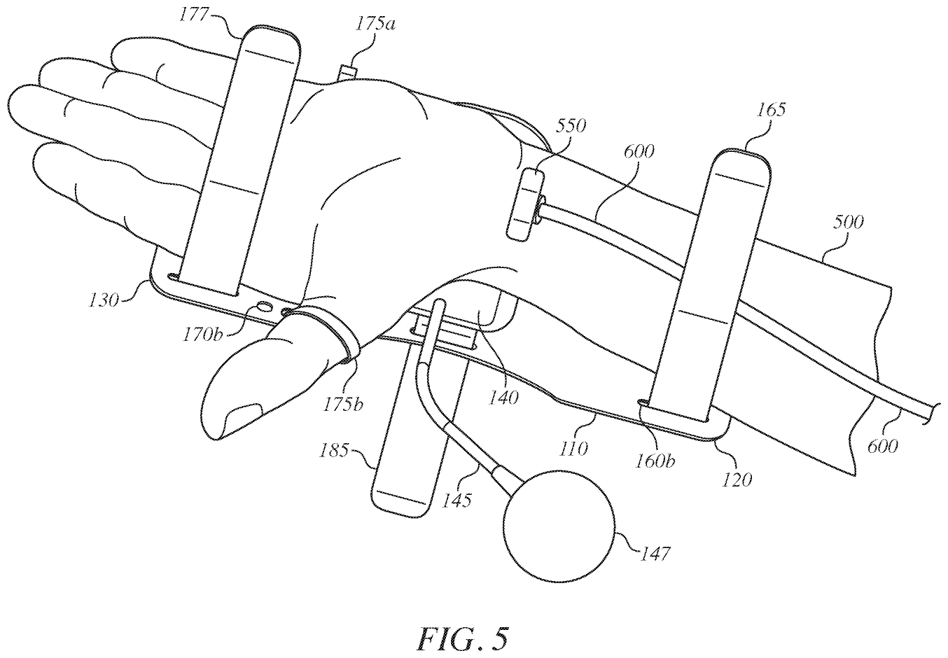

The inflatable means 140 when an air bladder further includes a plug or a tube for connection to a pump or other inflating mechanism or even a mouth for pushing of air into the inflatable means 140 when an air bladder. If the inflatable means 140 when an air bladder is to be connected to a pump via a tube 145 capable of connection to a pump such as a bulb or other manual or electronic pump or inflation means. For example, as shown in FIGS. 3 and 5 the tube 145 is connected to a manual hand pump 147 so as to inflate the inflatable means 140 when, an air bladder. It can be appreciated that a mechanical or automatic inflation pump (not shown) can also be attached to inflation port or tube 145 to inflate and deflate the inflatable means 140 when an air bladder. A vent valve (not shown) can also be incorporated into the inventive device 100 to allow a user to selectively deflate the inflatable means 140 when an air bladder. Further the inflatable means 140 when an air bladder may include a plug for inflation and deflation. The inflatable means 140 may be connected to a device including a preset inflation and deflation series such that the inflatable means 140 is inflated and deflated and so on over a period of time. Any know adjustable means may be employed beyond an air bladder including a mechanical or electrical adjustment system.

The inflatable means 140 when an air bladder is shown in one shape of a rounded rectangle in FIGS. 1 through 4 but any shape may be employed in the inventive inflatable wrist support 100. In other embodiments the shape may be a circle, rectangle, triangle, square, sphere, oval, semi-circle, donut, crescent, or any combination of shapes. The shape of the inflatable means 140 could be a triangle such that the wrist of a patient is supported by the point or top of the triangle. The upper portion of the inflatable means 140 when an air bladder, that portion which is on contact with the wrist when in use, may be contoured to mirror the shape of the wrist and hand or also contoured to provide traction against the wrist or hand or forearm and stability when in use.

The upper member when adjustable, such as an inflatable means 140, must be adjusted such that the exposed and extended upward facing wrist is at an angle sufficient for the user to locate the radial artery and perform the radial arterial cannulation while reducing many of the complications to the patient.

The inflatable means 140 when an air bladder and inflated may be sloped at an angle to support, secure, restrain and stabilize the forearm of a patient when the inventive inflatable wrist support 100 is in use. The sloped angle of the inflatable means 140 when an air bladder may be between about 10° to about 60°, preferably between about 30° to about 45°, when measuring the angle from the proximal end 120 of the elongated base member so as to support, secure and stabilize the forearm of a patient when the inventive inflatable wrist support 100 is in use. The sloped angle of the distal end of inflatable means 140 when an air bladder may be between about 20° to about 90°, preferably between about 60° to about 80, when measuring the angle from the proximal end 120 of the elongated base member (or between about 91° to about 160°, preferably between about 95° to about 125, when measuring the angle from the distal end 130 of the elongated base member). The angle differences are to further extend or hyperextend the wrist for better cannulation of the radial artery.

In another embodiment of the inflatable means 140 when an air bladder may include a concave portion (not shown) to support and secure the wrist of a patient when in use. The concave portion should be wide and long enough to support and secure the anatomy of the wrist without causing additional pain to the patient when in use. In another embodiment, not shown the inflatable means 140 when an air bladder may optionally include a malleable portion placed on top of the inflation means 140, possibly within the concave portion if included. The malleable portion may be comprised of a material such as foam, woven materials, gauze, cotton, padding and the like. This malleable portion may be disposable and changed out after each use of the inventive wrist support 100. The malleable portion may be adhered within the inflation means 140 with an adhesive or other fastening means.

The inventive inflatable wrist support 100 may also include a further fastening means to further secure either the fingers or palm of the hand of the patient when in use or to secure and stabilize the inserted atrial line when in use. As noted above, any movement of the arm or hand of the patient may compromise the integrity of the cannulation and also compromise the inserted atrial line during a subsequent medical procedure or therapy. Securing and stabilizing the inserted atrial line may thus be augmented by a third fastening means 165 while restrains the forearm and the radial arterial line (600).

A further embodiment of the present invention is shown in FIGS. 3 and 4 wherein a fourth fastening means 185 is shown capable of fastening to a table, board or surgical support during use for stabilization of the entire inventive inflatable wrist support 100. The fourth fastening means 185 may be a long strap which is connected to the base member 110 via apertures 180a, 180b, in one embodiment as shown located in the center of the base member 110. Another example of the forth fastening means 185 may be a strap with Velcro fasteners on each end when joined together to secure the inventive inflatable wrist support 100 to a table, board or surgical support when in use. The fourth fastening means 185 may be other known straps, an elastic band, a belt, or the like. The fourth fastening means 185 may be a combination of a flexible material with buckles, snaps Velcro of any commonly known fastener or locking means. Those skilled in the art may employ other known fastening means to secure and stabilize the inventive inflatable wrist support 100 when the inventive inflatable wrist support 100 is in use for radial arterial cannulation or intravenous line insertion.

The inventive inflatable wrist support 100 may be used in a hospital setting, a clinic of doctors office setting or even a home setting. The inventive inflatable wrist support 100 in use may be placed onto a flat surface such as a table, examining table or operating table. In one embodiment the inventive inflatable wrist support 100 may be affixed permanently to an operating table.

In use, the inventive inflatable wrist support 100 is placed on a surface such as a table. As seen in FIG. 5, the patient's forearm 500 is placed on the proximal end 120 of the elongated base 110, the wrist 550 is placed on the inflation means 140 (which is in an inflated state and optionally including a foam member for patient comfort and further securing of the wrist to limit movement), the upturned palm including fingers placed on the distal end 130 of the base member 110. The fastening means 165 is employed to secure and stabilize the forearm, the fastening means 175 is employed to secure and stabilize the fingers, and the thumb is then secured within the fastening means 175a or 175b depending on whether the right hand or left hand is being subject to the radial arterial cannulation. Thereafter the cannulation proceeds and a catheter 550 is inserted and an arterial line 600 is employed for any number of medical procedures such as cardiac catheterization, blood pressure monitoring or C.O.P.D. therapy. The adjustability of the wrist support 140 is used to adjust the height and angle of extension of the wrist in the palm facing upward position thus exposing the radial artery easier in a visual and touch sensitive status for the user to locate and insert the catheter 550 and radial line 600. Further, the fastening means 175a, 175b extends out or backward, possibly in a hyperextension position, of the thumb further exposing the radial artery for the user to locate the radial artery for insertion of the catheter 550 and radial line 600. The fastening means 177 stabilizes the fingers when in use assisting the user in conducting the radial arterial cannulation as the target radial artery is the wrist area needs to be restrained at the proper angle and flexion position to reduce the chance of improper insertion of a cannula or the catheter due to the patient's voluntary or involuntary movements. As the catheter 550 and needle combination, should be inserted into the patient's radial artery at the wrist at a 25°-50° angle to the skin in the long plane of the artery the adjustable and in this embodiment inflatable means 140 assists in obtaining the proper angle of the exposed wrist for the cannulation. As this insertion should be performed slowly and deliberately to maintain the proper angle of insertion and instead the natural reflexes in the wrist area of the patient and resulting spasms in the radial artery may compromise the cannulation so such reflexes and spasm, the wrist and fingers must be stabilized and even constrained to the extent possible during the radial arterial cannulation procedure. The components of the inventive wrist support meet these needs and assist the user in conducting a successful and less painful radial arterial cannulation on the patient. At any time after insertion of the radial line 600 the fastening means 165 may be loosened and then refastened such that the radial line 600 is restrained and secured under the fastening means 165.

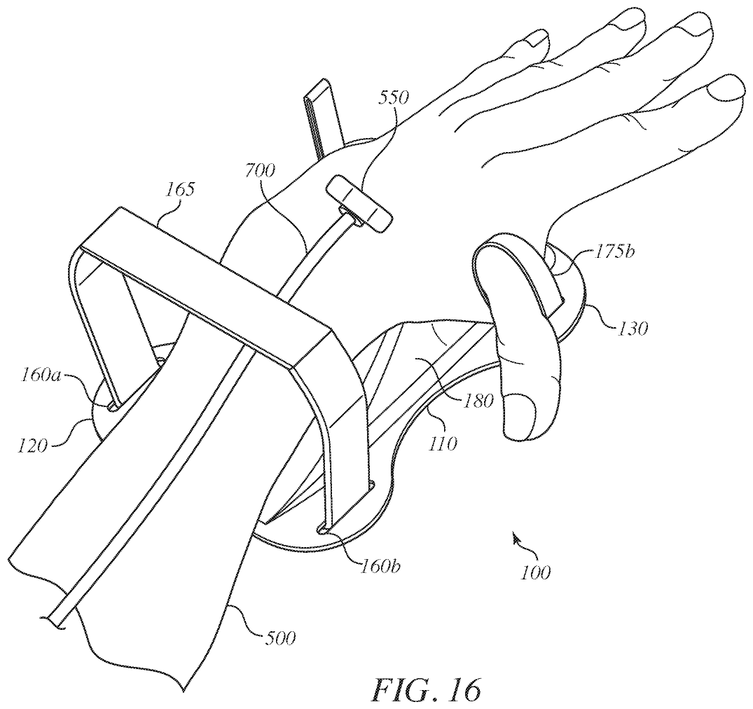

Another method of use is to insert an intravenous line wherein the same steps are taken but the patient's hand is placed on the wrist support 140 with the palm facing downward as shown in FIG. 16 with the catheter 550 inserted and an intravenous line 700 is employed for any number of medical procedures such as drug therapy or other uses.

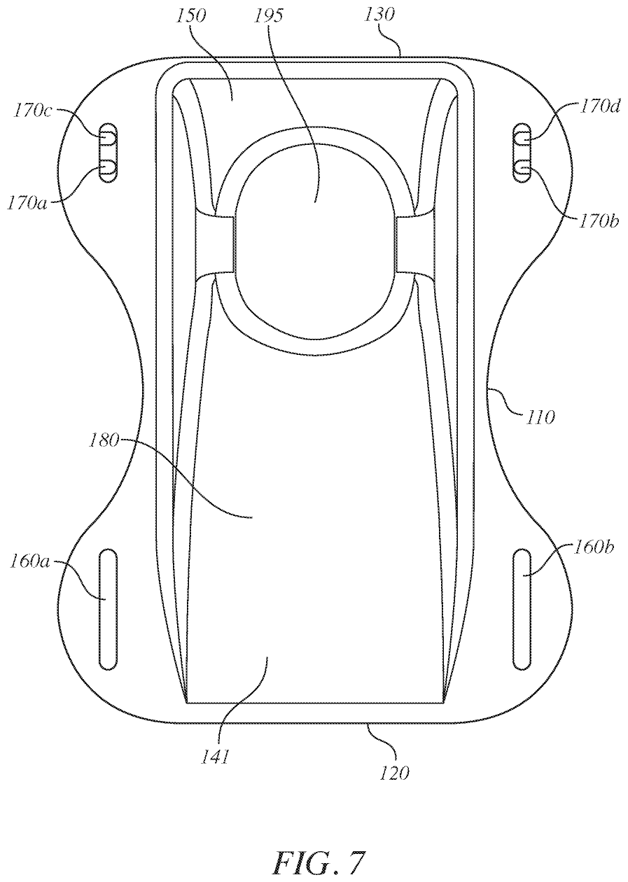

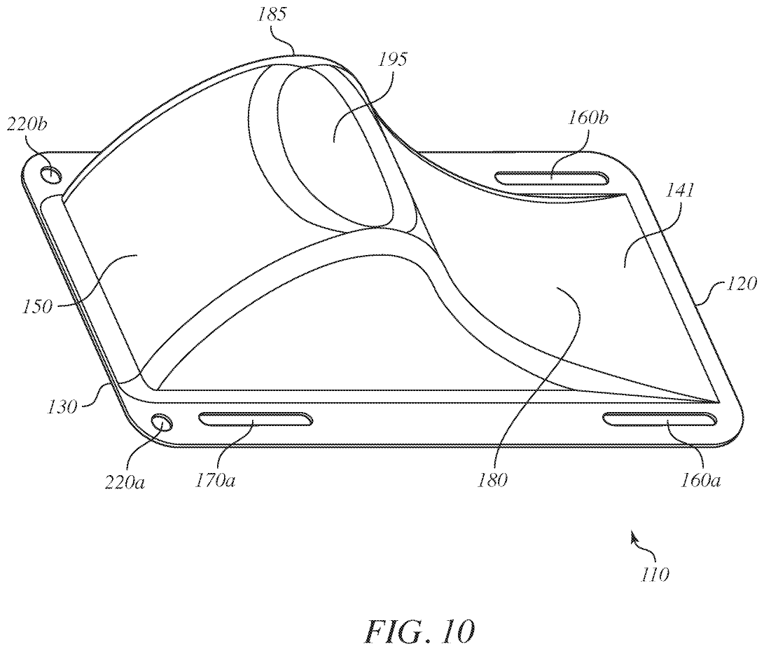

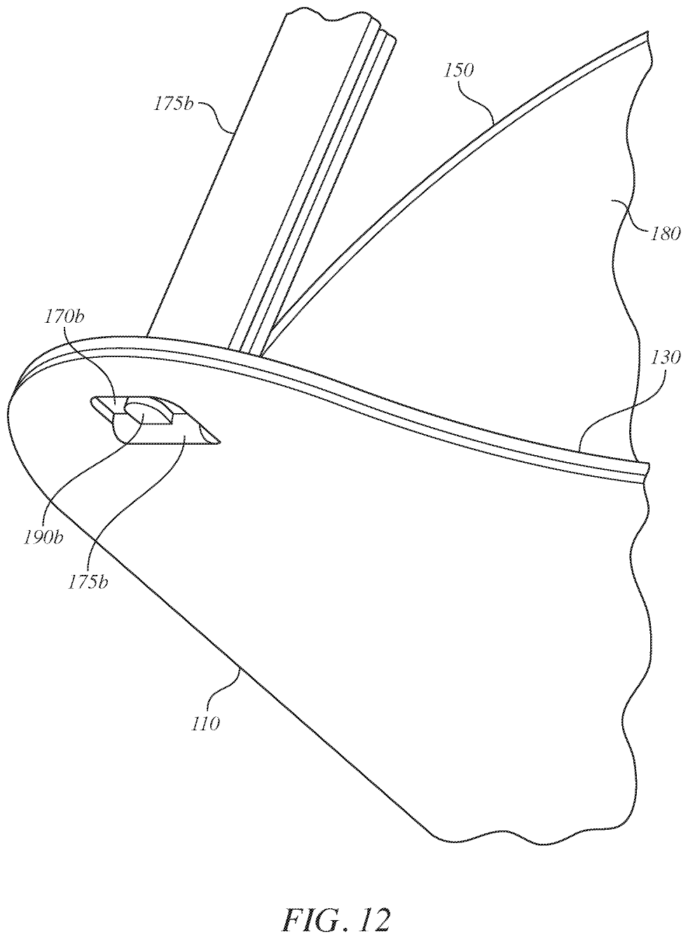

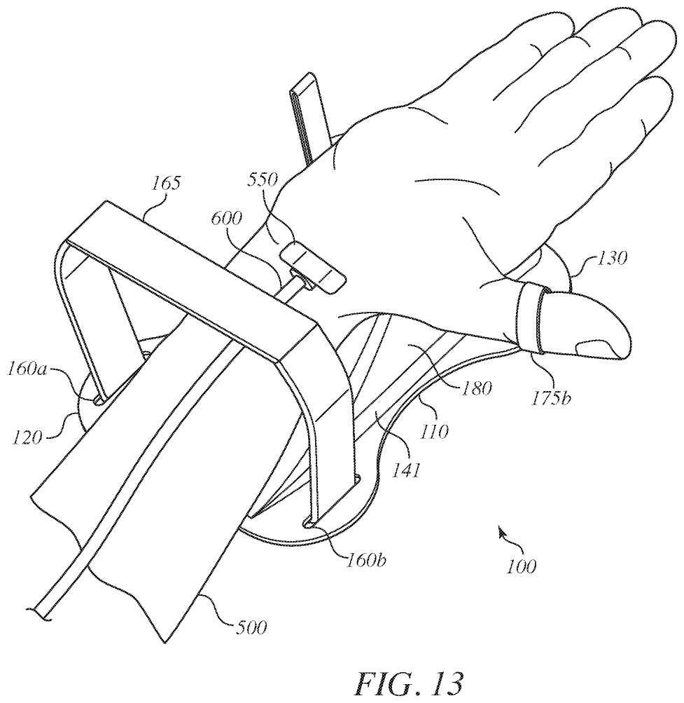

FIGS. 6 through 15 show other embodiments of the inventive wrist support 100 wherein the wrist support is not inflatable and is sloped such as an upper member 180 as shown in the Figures. Turning to FIG. 6 the inventive wrist support includes an elongated base member 110 having a proximal end 120 and a distal end 130. The proximal end includes at least two apertures 160a, 160b for insertion of a fastening means 165 to secure the forearm of a patient when the inventive wrist support is in use. The distal end 130 includes at least one aperture 170b for attachment to a fastening means 175b to secure the thumb of a patient when the inventive wrist support is in use. The elongated base member 110 may be comprised of any rigid material sufficient to support the wrist placed upon it. Suitable materials include without limitation polymers, plastics, foam, wood, metals and the like. The elongated base member 110 is preferably comprised of a polymer or plastic material for economic reasons and to make the inventive wrist support 100 easy to use by a medical professional when in operation. For commercialization and mass production, the to inventive wrist support 100 may formed of a suitable disposable material. In other embodiments the inventive wrist support 100 may be comprised a suitable rigid material capable of being sterilized and reusable, or capable of autoclaving, in this embodiment the fastening means 165, 175a would be disposable and, replaced after the sterilization of the previously used elongated base member 110 and upper member 180.

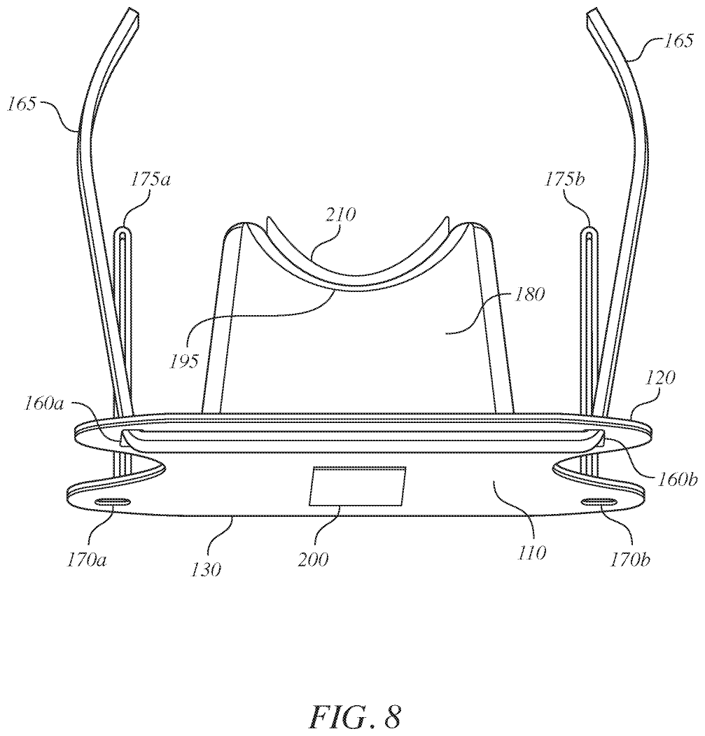

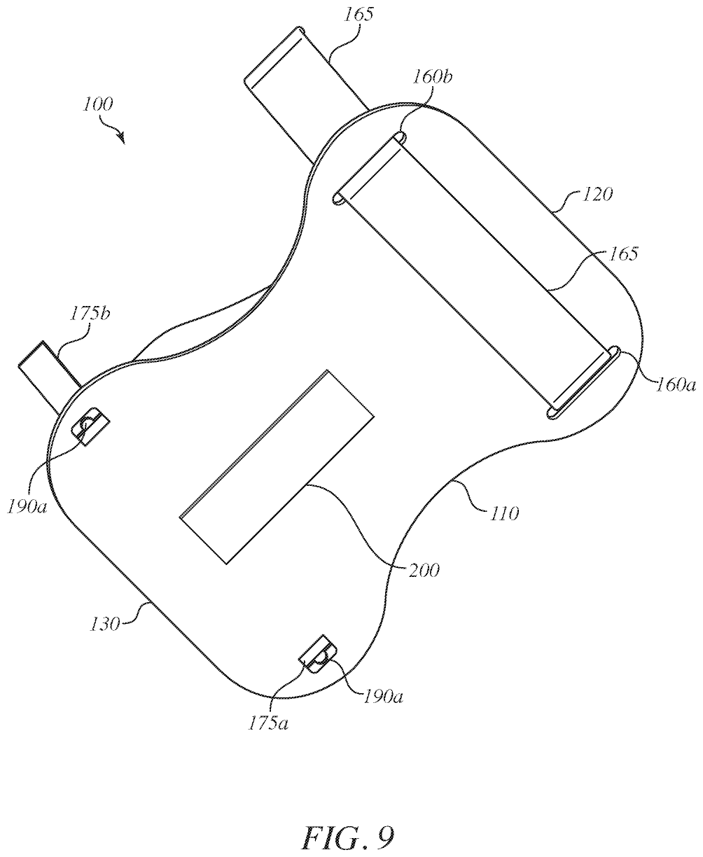

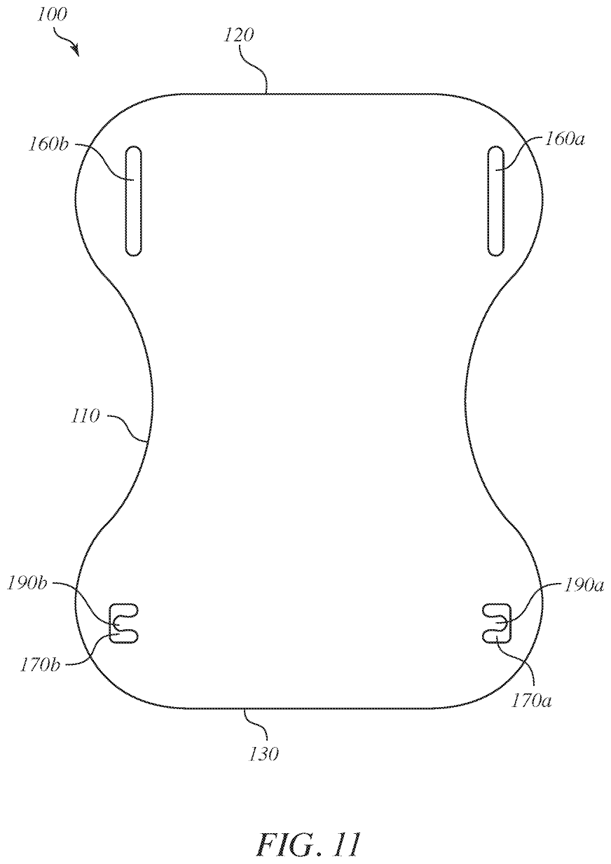

The bottom of the elongated base member 110 may optionally include, as seen in FIGS. 8 and 9, an attached non-skid member 200 which acts to stabilize the inventive wrist support 100 when placed on a metal surface, wood surface, plastic surface and other known surfaces. The non-skid member 200 may be attached to the bottom of the elongated base member 110 by an adhesive or other known fastening means. In FIG. 11 the bottom of the elongated base member 110 is shown without the attached non-skid member 200. In this embodiment of the present invention, four apertures (160a, 160b, 170a, 170b) are shown in FIG. 11, which shows the bottom of the elongated base member 110 without any of the fastening means so as to show the tabs 190a, 190b which are used to attach the fastening means 175a, 175b. The fastening means 175a, 175b may be rubber constraints such as rubber bands, which are hooked individually onto each of the tabs 190a, 190b. In another embodiment of the present invention only one aperture 170a is used with one tab 190a and one fastening means 175a or in the alternative aperture 170b is used with one tab 190b and one fastening means 175b, the choice depending on the right or left hand of the patient such that the one fastening means (either 175a or 175b) secures and hyperextends the thumb of the patient. Such choice will depend on whether the radial arterial cannulation is being performed on the right wrist or left wrist. It is preferred that the elongated base member 110 include two apertures 170a, 170b so that the inventive wrist support 100 is universal and can be used with both the right hand wrist and left hand wrist. Other known fastening means 175a, 175b include straps, loops, gloves and the like. Those skilled in the art may employ other known fastening means to secure and hyperextend the thumb of the patient when the inventive wrist support 100 is in use for radial arterial cannulation or intravenous line insertion and the subsequent medical procedures for each process.

In this embodiment of the present invention, the elongated base member 110 also includes at least two apertures 160a, 160b at the proximal end 120. A second fastening means 165 is inserted within each of the apertures 160a, 160b and is looped under the bottom of the inventive wrist support 100 as shown in FIGS. 8 and 9. Each end of the second fastening means 165 is located above the top of the elongated base member and in use fastens together to secure the forearm of the patient when in use. The fastening means 165 should provide a secure fit and restraint of the patient's arm and hand. One example of the fastening means 165 may be a strap with Velcro fasteners on each end when joined together to secure the forearm of the patient. The fastening means 165 may be other known straps, an elastic band, a belt, or the like. The fastening means 165 may be a combination of a flexible material with buckles, snaps Velcro of any commonly known fastener or locking means. Those skilled in the art may employ other known fastening means to secure and stabilize the forearm of the patient when the inventive wrist support 100 is in use for radial arterial cannulation. Stabilization is important to control the artery and wrist during the cannulation as well as stabilizing the inserted radial arterial line during any subsequent medical procedure as well as an intravenous line insertion procedure and subsequent therapy or medical procedures.

The elongated base member 110 is attached to an upper member 180. The upper member 180 is slightly smaller in length and width than the elongated base member 110 for stability of the inventive wrist support 100 as well as providing for the apertures (160a, 160b, 170a, 170b) as seen in FIGS. 6-16. The upper member 180 has a proximal end 141 and a distal end 150 both sloped upwards toward a middle portion 185. The proximal end 141 of the upper member 180 is at a sloped angle to support, secure and stabilize the forearm of a patient when the inventive wrist support 100 is in use. The sloped angle of the proximal end 141 of the upper member 180 may be between about 10° to about 60°, preferably between about 30° to about 45°, when measuring the angle from the proximal end 120 of the elongated base member (see 300). The proximal end 141 of the upper member 180 is at a sloped angle to support, secure and stabilize the forearm of a patient when the inventive wrist support 100 is in use. The sloped angle of the distal end 150 of the upper member 180 may be between about 20° to about 90°, preferably between, about 60° to about 80, when measuring the angle from the proximal end 120 of the elongated base member (see 400) (or between about 91° to about 160°, preferably between about 95° to about 125, when measuring the angle from the distal end 150 of the elongated base member). Generally, the angle 400 will be larger than the angle 300, in some instances at least double. The angle differences are to further extend or hyperextend the wrist for better cannulation of the radial artery as seen in FIGS. 6-8. 10, and 12-16.

In the upper member 180 the middle portion 185 includes a concave portion 195 to support and secure the wrist of a patient when in use. The concave portion 195 should be wide and long, enough to support and secure the anatomy of the wrist without causing additional pain to the patient when in use. As seen in FIGS. 6 and 8, the inventive wrist, support 100 may optionally include a malleable portion 210 placed within the concave portion 195 of the upper member 180. The malleable portion 210 may be comprised of a material such as foam, woven materials, padding and the like. The malleable portion 210 may be adhered within the concave portion 195 with an adhesive or other fastening means. This malleable portion 210 may be disposable and changed out after each use of the inventive wrist support 100.

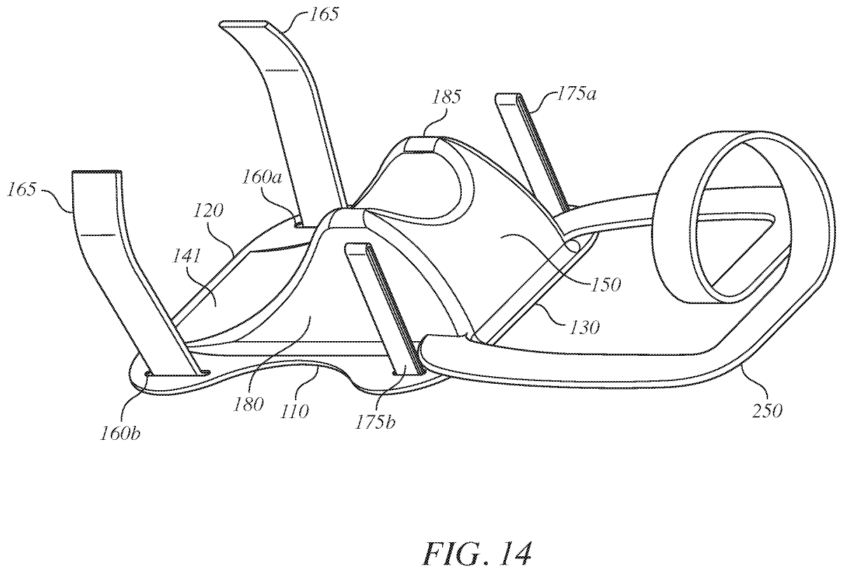

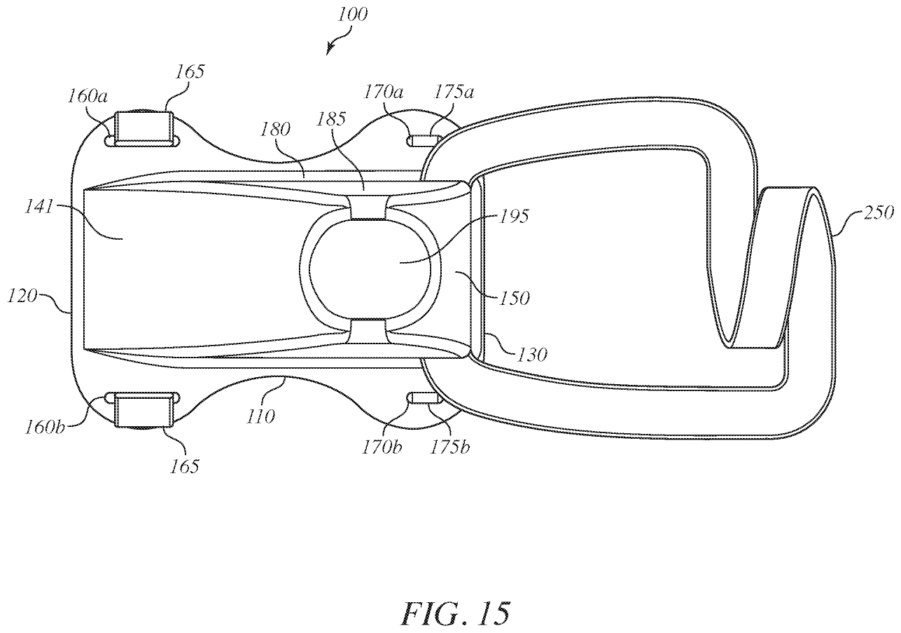

The inventive wrist support 100 may also include a further fastening means to further secure either the fingers or palm of the hand of the patient when in use or to secure and stabilize the inserted atrial line when in use. As noted above, any movement of the arm or hand of the patient may compromise the integrity of the cannulation and the inserted atrial line. Securing and stabilizing the inserted atrial line may thus be augmented by a third fastening, means 250 as shown in FIGS. 14 and 15. The third fastening means 250 may be a long strap which is connected to the base member 110 or the distal end 150 of the upper member 180. Another example of the third fastening means 250 may be a strap with Velcro fasteners on each end when joined together to secure the forearm of the patient. The third fastening means 250 may be other known straps, an elastic band, a belt, or the like. The third fastening means 250 may be a combination of a flexible material with buckles, snaps Velcro of any commonly known fastener or locking means. Those skilled in the art may employ other known fastening means to secure and stabilize the fingers and palm or hand of the patient when the inventive wrist support 100 is in use for radial arterial cannulation.

In yet another embodiment of the invention the upper member 180 may be rigid or semi-rigid or may be adjustable in height and resulting adjustable angle of the wrist when in use with the inventive wrist support 100. The upper member 180 may be comprised of any rigid material sufficient to support the arm and hand placed upon it. Suitable materials include without limitation polymers, plastics, foam, wood, metals and the like. The upper member 180 is preferably comprised of a polymer or plastic material for economic reasons and to make the inventive wrist support 100 easy to use by a medical professional when in operation. For commercialization and mass production, the inventive wrist support 100 may formed of a suitable disposable material. In other embodiments the inventive wrist support 100 may be comprised a suitable rigid material capable of being sterilized and reusable, or capable of autoclaving, and in this embodiment the fastening means 165, 175, 177 would be disposable and replaced after the sterilization of the previously used upper member 180. In another embodiment the upper member 180 may be comprised of a semi-rigid material.

In yet another embodiment of the invention the upper member 180 may be adjustable in height and resulting adjustable angle of the wrist when in use with the inventive wrist support 100. The upper member 180 may be adjusted via any known adjusting means such as a ratchet, lock, screw thread or the like. The upper member 180 when in use on a patient may be adjusted upward for a more extended angle of the wrist, such that the upper wrist forms a reflex angle and exposes the wrist for radial arterial cannulation. The upper member 180 may be adjusted downward so that the wrist is, not extended in such an angle and relaxed after the initial arterial cannulation and the line has been established and placed in the patient's wrist. The upper member 180 can thereafter be adjusted upward or downward or adjusted in angle as needed by the user for the patient's wrist extension or angle.

In other embodiments of the invention an indicator graphic may be included outlining a hand to show that the fingers are placed on the distal end 130 of the elongated base member 110 and the forearm rests on the proximal end 120 of the elongated base member 110. Other graphics such as phrases of “Place Wrist Here” or “Place Fingers Here” may be used or an arrow pointing towards the distal end 130 of the elongated base member 110 or other indicator graphics as possible. Since the inventive wrist support 100 in use is ambidextrous in that it may be used with either the left or right hand, the word graphics may be used.

The invention encompasses a kit including the inventive wrist support 100 and a catheter and needle. Other embodiments include a needle guide such as that disclosed in PCT/US14/59576 by Dr. Ravikumar (Vein Access Needle Guide Assembly and Methods of Use) which is incorporated by reference herein in its entirety, and Ser. No. 14/354,243 filed Jun. 16, 2015 which is incorporated by reference herein in its entirety, and Serial No. 14/ 886,765 filed Oct.r 15, 2015 which is incorporated by reference herein in its entirety. The method of use of the inventive wrist support 100 may further include use of the inventive needle guide as disclosed in such applications.

The present invention obviates the disadvantages associated with the traditional arm boards and/or wrist supports by providing an adjustable wrist support so as to vary the angle of flexion of the exposed wrist in order for the user to obtain a better or even optimal angle for insertion of a catheter during radial arterial cannulation and this reducing complications of such procedure.

Another embodiment of the inventive wrist support 100 includes a preformed unitary support which can be readily and inexpensively manufactured by a simple molding process.

The inventive wrist support 100 provides better access to the radial artery due to the angle of the wrist and the hyperextension of the thumb. Further, the inventive wrist support provides increased stabilization of the inserted atrial line and/or catheter when in use based on the stabilization of the forearm and the wrist and hand during use.

The inventive wrist support 100 may be adjustable (140 or 180) so that the angle of the wrist may be adjusted for insertion of either a radial arterial line or an intravenous line but then the angle is decreased so that the wrist is more relaxed during a subsequent medical procedure or therapy such as C.O.P.D therapy and chemotherapy. The adjustable wrist support 100 may then be adjusted for an increased angle of the wrist and such adjustment may be increased or decreased throughout the is medical procedure while the hand, wrist and forearm are stabilized through the various fastening means employed with the inventive wrist support 100.

The inventive wrist support 100 may be inflated so as to adjust the angel of the wrist during the arterial cannulation or intravenous line insertion. The inflation means may be adjusted so as to lower or raise the wrist and extend further or lessen the extension of the wrist and exposure of the radial artery or veins and flexion of the wrist.

The inventive wrist support 100 may reduce decannulation of the intra-arterial line and thus reduce the risk of blood to flow out of the radial artery at systolic pressure, which could lead to a large hematoma or in a worst case scenario if left undetected and untreated may cause a person to bleed to death. The inventive wrist support 100 may reduce pain and complications to the patient during the radial arterial cannulation or intravenous line insertion procedures. The inventive wrist support 100 may reduce the need for repeated cannulation and thus reduce other complications such as a hematoma, blood loss, pain, infection and extended post-operative-time and impairments.

The inventive wrist support 100 in use hyperextends the thumb separately from the remaining four fingers and upturned palm providing a better positioned radial artery which is exposed at an angle for insertion of the cannula or arterial line and may result in a better maintained and secure manner throughout the medical procedure which may reduce complications to the patient. The inventive wrist support 100 when adjustable provides a better positioned radial artery which is exposed at an angle for insertion of the cannula or arterial line and this may reduce complications to the patient.

The inventive wrist support 100 is an economic, convenient and effective wrist support apparatus for supporting a patient's wrist and forearm during radial arterial cannulation, intravenous line insertion and subsequent medical procedures or therapy.

Although the exemplary embodiments of the present invention have been is described, it is understood that the present invention should not be limited to these exemplary embodiments but various changes and modifications can be made by one ordinary skilled in the art within the spirit and scope of the present invention as hereinafter claimed. The invention has been described in terms of embodiments thereof, but is more broadly applicable as will be understood by those skilled in the art. The scope of the invention is only limited by the following claims.

We claim:

1. A wrist support for positioning and restraining a patient's wrist comprising:
an elongated base member including at least a pair of apertures on each side of a distal end of the elongated base member and at least a pair of apertures on each side of a proximal end of the elongated base member;
a fastening means inserted within each aperture on the distal end of the elongated base member and secured below the elongated base member;
a second fastening means inserted within each aperture on the proximal end of the elongated base member and secured below the elongated base member;
a third pair of apertures on the elongated base member located closer to the center of the elongated base member from the pair of apertures located on the distal end of the elongated base member and a third fastening means inserted within one of the third pair of apertures; and
an adjustable means located in the center of the elongated base member.

2. The wrist support of claim 1 wherein the adjustable means is an inflatable means.

3. The wrist support of claim 2 wherein the inflatable means is selected from a manual air system, a mechanical inflatable system, or an electronic inflatable system.

4. The wrist support of claim 1 wherein the adjustable means further compromises an air bladder, tube and pump.

5. The wrist support of claim 4 wherein the inflatable means further compromises a valve to release air within the air bladder.

6. The wrist support of claim 1 wherein the first and second fastening means are flexible straps.

7. The wrist support of claim 6, wherein the straps have hook and loop fasteners or belts.

8. The wrist support of claim 1 wherein the third fastening means is a flexible band.

9. The wrist support of claim 1, wherein the inflatable means is in the inflated position and a patient's forearm and hand are positionable, palm up, on the inflatable means for receiving a radial atrial line, with the patient's wrist extended and exposed for the radial atrial line.

10. The wrist support of claim 1 further comprising a fourth pair of apertures each located near the side edge of a center portion of the elongated base and a fourth fastening means inserted within each aperture on the center portion of the elongated base and secured below the elongated base.

11. The wrist support of claim 10 wherein the fourth fastening means is a flexible strap.

12. The wrist support of claim 11, wherein the strap has hook and loop fasteners or belts.

13. A wrist support for positioning and restraining a patient's wrist during a radial arterial cannulation comprising:
an elongated base member including at least a pair of apertures on each side of a distal end of the elongated base and at least a second pair of apertures on each side of a proximal end of the elongated base, and at least a third pair of apertures on each side of a center portion of the elongated base;
a fastening means inserted within each aperture on the distal end of the elongated base and secured below the elongated base configured to restrain a patient's fingers during the cannulation;
a second fastening means inserted within each aperture on the proximal, end of the elongated base and secured below the elongated base configured to restrain a patient's forearms during the cannulation;
a third fastening means inserted within each, aperture on the center portion of the elongated base and secured below the elongated base configured to secure the wrist support to an immobile object during the cannulation;
a fourth pair of apertures on the elongated base located closer to the center of the elongated base from the pair of apertures located on the distal end of the elongated base and a fourth fastening means inserted within one of the fourth pair of apertures configured to restrain a patient's thumb during the cannulation; and
an adjustable means located in the center of the elongated base configured to start in pone position and to extend the patient's wrist during the cannulation when the adjustable means is in a second position.

14. The wrist support of claim 13 wherein the adjustable means is an inflatable means selected from a manual air system, a mechanical inflatable system, or an electronic inflatable system.

15. The wrist support of claim 13 wherein the adjustable means compromises an air bladder, tube and pump.

16. The wrist support of claim 15 wherein the inflatable means further compromises a valve to release air within the air bladder.

17. The wrist support of claim 13 wherein the first, second and third fastening means are flexible straps.

18. The wrist support of claim 1 wherein the straps have hook and loop fasteners or belts.

19. The wrist support of claim 13 wherein the fourth fastening means is a flexible band.

20. A method for positioning and restraining a patient's wrist during a radial arterial cannulation comprising:
providing a wrist support apparatus having (a) an elongated base member including at least a pair of apertures on each side of a distal end of the elongated base member and at least a pair of apertures on each side of a proximal end of the elongated base member, (b) a fastening means inserted within each aperture on the distal end of the elongated base member and secured below the elongated base member, (c) a second fastening means inserted within each aperture on the proximal end of the elongated base member and secured below the elongated base member, (d) a third pair of apertures on the elongated base member located closer to the center of the elongated base member from the pair of apertures located on the distal end of the elongated base member and a third fastening means inserted within one of the third pair of apertures, and (e) an adjustable means located in the center of the elongated base member;
placing the patient's wrist in a palm up position on the adjustable means of the elongated base member with the patient's forearm on the proximal end of the elongated base member and the patient's fingers on the distal end of the elongated base member;
adjusting the adjustable means to provide a sufficient angle of flexation of the patient's wrist to insert a radial arterial line during the cannulation;
securing the first fastening means on the patient's fingers;
securing the second fastening means on the patient's forearm;
securing the third fastening means on the patient's thumb so that the thumb is in an extended position;
locating the radial artery; and
inserting a needle and catheter into the patient's a grist in the radial artery.
